(19) United States
(12) Patent Application Publication
(10) Pub. No.: US 2013/0150317 A1
(43) Pub. Date: Jun. 13, 2013

(54) COMPOUNDS FOR THE TREATMENT OF RENAL CELL CARCINOMA

(71) Applicants: Eamonn Maher, Birmingham (GB); Xiaohong Lu, Birmingham (GB)

(72) Inventors: Eamonn Maher, Birmingham (GB); Xiaohong Lu, Birmingham (GB)

(73) Assignee: Myrovlytis Technology Ventures Limited, London (GB)

(21) Appl. No.: 13/579,420

(22) PCT Filed: Feb. 17, 2011
(86) PCT No.: PCT/GB11/50313

§ 371 Date: Sep. 14, 2012

(30) Foreign Application Priority Data
Feb. 18, 2010 (GB) 1002773.8

(51) Int. Cl. A61K 31/704; A61K 31/436; C07H 15/24

(52) U.S. Cl. CPC A61K 31/704; C07H 15/24; A61K 31/436
USPC 514/33; 536/18.1

(57) ABSTRACT

A composition comprising mithramycin or a pharmaceutically acceptable salt or solvate thereof for use in the treatment of renal cell carcinoma. In particular, the composition is suitable for use in the treatment of clear cell renal cell carcinoma. The composition is particularly useful for use in the treatment of renal cell carcinoma associated with Von Hippel-Lindau disease or Birt-Hogg-Dubé syndrome. A composition comprising mithramycin or a pharmaceutically acceptable salt or solvate thereof for use as a cytotoxic agent against FLCN-null or VHL-null renal cell carcinoma cells.

This invention relates to drugs for use in the treatment of diseases. More particularly, this invention relates to compositions, including compositions comprising mithramycin, for use in the treatment of renal cell carcinoma including clear cell renal cell carcinoma, Von Hippel-Lindau disease or Birt-Hogg-Dubé syndrome. This invention also relates to the use of mithramycin for the manufacture of a medicament for the treatment of renal cell carcinoma.

Mithramycin (also known as aurelic acid, plicamycin or mitramycin) is an aureolic-acid type polyketide antibiotic produced by various soil bacteria of the genus Streptomyces. Mithramycin has formula:

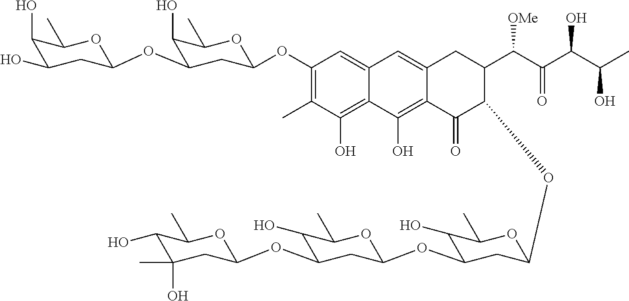 [SMILES: CO[C@H](C(=O)[C@@H](O)[C@@H](C)O)C1Cc2cc3cc(OC4CC(OC5CC(O)C(O)C(C)O5)C(O)C(C)O4)c(C)c(O)c3c(O)c2C(=O)[C@H]1OC1CC(OC2CC(OC3CC(C)(O)C(O)C(C)O3)C(O)C(C)O2)C(O)C(C)O1]

Mithramycin has been used as a targeted therapy to treat hypercalcaemia in patients with bone metastases, Paget's disease, testicular carcinoma and leukaemia (Yuan et al, Cancer 2007; 110: 2682-2690). It has also been shown that mithramycin has potential as a neuroprotective drug for the alleviation of symptoms associated with β-thalassemia and sickle cell anaemia.

Mithramcyin binds to GC-rich regions in the minor groove of DNA and inhibits the transcription of genes with GC-rich promoters. Mithramycin therefore inhibits transcription of genes regulated by transcription factors that bind to such sequences, such as the Sp1 family. Sp1 has been shown to be involved in the regulation of the angiogenesis stimulator vascular endothelial growth factor (VEGF), and the use of mithramycin for the inhibition of angiogenesis in mammals has been reported.

Renal cell carcinoma (RCC) accounts for 2-3% of all cancers and is the most common type of kidney cancer in adults. RCC is a heterogeneous disorder with a number of histopathological subtypes, although conventional clear cell RCC (ccRCC) accounts for more than 75% of cases of RCC. Non-clear-cell forms of RCC comprise papillary (or chromophil) RCC, chromophobe tumours, oncocytoma, collecting duct carcinoma and the rare medullary carcinoma.

Surgical resection is currently the preferred treatment for locally confined RCC and can often achieve a cure in the earlier stages of RCC. RCC has traditionally been considered to be largely resistant to radiotherapy and in vitro studies have shown that renal cancer cells are among the least radiosensitive of human cell types. Furthermore, the majority of advanced RCC tumours have proved to be resistant to cytotoxic agents and therefore chemotherapy has had a very limited role in the treatment of metastatic renal cancer.

Most cases of RCC are sporadic and only about 3% of all cases have a genetic cause. However, investigations into rare inherited forms of RCC have provided seminal insights into the molecular pathogenesis of both familial and sporadic RCC.

Von Hippel-Lindau (VHL) disease is a dominantly inherited multisystem familial cancer syndrome characterised by the development of clear cell renal cell carcinoma (ccRCC) as well as haemangioblastomas, pancreatic lesions and phaeochromocytoma. VHL is the most common cause of inherited RCC.

The identification of the gene for VHL disease has led to recognition that the most frequent genetic event in the evolution of sporadic ccRCC is somatic inactivation of the VHL tumour suppressor gene (TSG). Further work has led to an understanding that VHL TSG inactivation leads to dysregulation of the HIF-1 and HIF-2 transcription factors and activation of hypoxia-responsive gene pathways (Latif et al., Science 1994; 260:1317-20; Foster et al., Cancer 1994; 69:230-4; Gnarra et al., Nat Genet. 1994; 7:85-90; Clifford et al., Genes Chromosomes Cancer 1998; 22:200-9; Maxwell et al., Nature 399:271-275, 1999; Banks et al., Cancer Res 2006; 66:2000-7).

It is now known that under normoxic conditions, the VHL tumour suppressor gene product, pVHL, functions in a ubiquitin ligase complex that targets hypoxia-response transcription factor subunits (HIF-1α and HIF-2α) for destruction in the proteasome. VHL inactivation results in elevated levels of HIF-1 and HIF-2, leading to overexpression of target genes involved in growth and angiogenesis, such as VEGF and PDGF.

These findings have provided a rationale for the use of drugs such as sorafenib and sunitinib (inhibitors of HIF target gene pathways) in the treatment of metastatic RCC (Patel et al., Br J. Cancer. 2006; 94:614-9; Motzer et al., J Clin Oncol. 2009; 27:3584-90).

Birt-Hogg-Dubé (BHD) syndrome is another dominantly inherited familial cancer syndrome associated with susceptibility to RCC. BHD is also associated with benign skin fibrofolliculomas (hamartomatous tumours of the hair follicle) and multiple lung cysts and spontaneous pneumothrorax (Toro et al., J. Med. Genet. 2008; 45: 321-331; BHD-associated renal tumours are of variable histopathology but are often chromophobe RCC/oncocytoma.

BHD syndrome results from inactivating mutations in the folliculin (FLCN) gene (Nickerson et al., Cancer Cell 2002; 2: 157-164; Schmidt et al., Am J Hum Genet. 2005; 76: 1023-33; Lim et al., Hum Mutat. 2010 Jan. 31(1):E1043-51) and renal tumours from BHD patients demonstrate somatic FLCN loss. The precise function of the FLCN gene product is still being elucidated, but folliculin (and the folliculin interacting proteins FNIP1 and FNIP2) have been linked to the mTOR and AMPK signalling pathways (Baba et al., Proc. Natl. Acad. Sci. U.S.A. 2006; 103: 15552-15557; Hasumi et al., Gene 2008; 415: 60-7; Takagi et al., Oncogene 2008; 27: 5339-47). In mice with kidney-targeted homozygous inactivation of Flcn, renal tumours and cysts developed with activation of mTOR and the mTOR inhibitor rapamycin diminished kidney pathology and increased survival (Baba et al., J. Natl. Cancer Inst. 2008; 100: 140-154; Chen et al., PloS ONE 2008; 33: e3581). mTOR inhibitor drugs (e.g. Temsirolimus, Everolimus, etc) have shown promise as treatments for metastatic RCC (Molina and Motzer Clin Genitourin Cancer 2008 December; 6 Suppl 1:57-13).

Patients with BHD syndrome are typically offered renal imaging to facilitate early detection of RCC. However, some patients may only be diagnosed after presentation with advanced RCC. Treatment of metastatic RCC is challenging for both familial and sporadic cases. Although occasional patients may respond to immunotherapy with the cytokines interferon and interleukin-2, recently treatment with targeted therapies to HIF downstream targets (e.g. Sunitinib, Sorafenib, Bevacizumab, etc) and the mTOR pathway (e.g. Temsirolimus, Everolimus) has emerged as the most frequent management strategy. However these agents, whilst prolonging life, are not cytotoxic and so the identification of targeted cytotoxic agents would be a significant advance. Chromomycin A3 (ChA3) (an aureolic acid compound) has been identified as a HIF-dependent cytotoxin. ChA3 shows discriminate killing of VHL-deficient cells in ccRCC cell lines (Sutphin et al., Cancer Res 2007; 67 (12); 5896-5902). It has been shown that overexpression of HIF-2α In VHL-positive clear cell RCC cell lines phenocopies the effect of VHL inactivation on susceptibility to ChA3 toxicity. However, ChA3 does not show differential growth inhibitory activity in FLCN-deficient and FLCN-wild type cell lines suggesting it is not likely to be a useful as drug treatment for BHD syndrome.

There is a need for a treatment for renal cell carcinoma that overcomes the disadvantages discussed above. Furthermore, there is a need for a treatment that targets cells deficient in the FLCN gene and diseases associated with such defects.

### SUMMARY OF INVENTION

According to the present invention there is now provided a composition comprising mithramycin or a pharmaceutically acceptable salt or solvate thereof for use in the treatment of renal cell carcinoma.

In particular, the composition is suitable for use in the treatment of clear cell renal cell carcinoma.

The composition is particularly useful for use in the treatment of renal cell carcinoma associated with Von Hippel-Lindau disease or Birt-Hogg-Dubé syndrome.

Also provided according to the present invention is a composition comprising mithramycin or a pharmaceutically acceptable salt or solvate thereof for use as a cytotoxic agent against FLCN-null or VHL-null renal cell carcinoma cells.

Cytotoxic agents are agents that are toxic to cells and can lead to a variety of outcomes for cells. Cells may stop actively growing and dividing, or may undergo necrosis, or the cells may undergo programmed cell death (apoptosis).

Cells undergoing necrosis lose membrane integrity, exhibit rapid swelling, shut down metabolism and release the cell contents into their surroundings. The process of apoptosis is an ordered sequence of events characterised by a change in refractive index, cytoplasmic shrinkage, nuclear condensation and DNA cleavage. Apoptotic cells shut down metabolism, lose membrane integrity and lyse.

Also provided according to the present invention is a composition comprising mithramycin or a pharmaceutically acceptable salt or solvate thereof for use in the inhibition of growth of FLCN-null renal cell carcinoma cells.

Also provided according to the present invention is a composition comprising mithramycin or a pharmaceutically acceptable salt or solvate thereof for use in the Inhibition of growth of VHL-null renal cell carcinoma cells.

The term “inhibiting” means decreasing, slowing or stopping. Thus, a compound of this invention can decrease, slow or stop the growth of a tumour cell. As used herein, “growth” means increase in size or proliferation or both. Thus, a compound of this invention can inhibit a tumour cell from becoming larger and/or can prevent the tumour cell from dividing and replicating and increasing the number of tumour cells.

A cell can be in vitro. Alternatively, a cell can be in vivo and can be found in a subject. A “cell” can be a cell from any organism including, but not limited to, a bacterium.

Also provided is a composition comprising mithramycin or a pharmaceutically acceptable salt or solvate thereof for use in inducing the death of FLCN-null renal cell carcinoma cells.

Also provided is a composition comprising mithramycin or a pharmaceutically acceptable salt or solvate thereof for use in inducing the death of VHL-null renal cell carcinoma cells.

Also provided is a composition comprising mithramycin or a pharmaceutically acceptable salt or solvate thereof for use in the treatment of renal cell carcinoma associated with FLCN inactivation.

The invention also provides a composition comprising vincristine or a pharmaceutically acceptable salt or solvate thereof for use in the treatment of renal cell carcinoma associated with FLCN inactivation.

The invention also provides a composition comprising paclitaxel (taxol) or a pharmaceutically acceptable sale or solvate thereof for use in the treatment of renal cell carcinoma associated with FLCN inactivation.

The invention also provides a composition comprising phyllanthoside or a pharmaceutically acceptable sale or solvate thereof for use in the treatment of renal cell carcinoma associated with FLCN inactivation.

Genes may be inactivated by genetic mutation. Mutations may include point mutations (transitions or transversions), insertions or deletions. Point mutations may be silent (code for the same amino acid), missense (code for a different amino acid) or nonsense (code for a stop). Insertions may alter splicing of the mRNA or cause a frameshift altering the gene product. Deletions may also alter the reading frame thereby affecting the gene product.

On a larger scale, mutations in chromosomal structure can include amplifications or gene duplications, deletions of chromosomal regions, chromosomal translocations, interstitial deletions and chromosomal inversions.

Also provided is a composition comprising mithramycin or a pharmaceutically acceptable salt or solvate thereof for use in the treatment of renal cell carcinoma associated with VHL inactivation.

Also provided is a composition comprising mithramycin or a pharmaceutically acceptable salt or solvate thereof for us in the differential growth inhibition of FLCN-null cells over FLCN-wild type cells.

Preferably the composition comprises mithramycin or a therapeutically effective derivative or metabolite thereof.

Preferably, the composition further comprises rapamycin or a pharmaceutically acceptable salt or solvate thereof. The composition may also comprise a therapeutically effective derivative or metabolite of rapamycin.

Also provided according to the invention is use of mithramycin or a pharmaceutically acceptable salt or solvate thereof for the manufacture of a medicament for the treatment of renal cell carcinoma.

In particular, there is provided use of mithramycin or a pharmaceutically acceptable salt or solvate thereof for the manufacture of a medicament for the treatment of renal cell carcinoma.

There is also provided use of mithramycin or a pharmaceutically acceptable salt or solvate thereof for the manufacture of a medicament for the treatment of Von Hippel-Lindau disease or Birt-Hogg-Dubé syndrome.

The invention also provides a method of treating renal cell carcinoma comprising administering to a subject a composition comprising mithramycin or a pharmaceutically acceptable salt or solvate thereof.

The invention also provides a method of treating renal cell carcinoma comprising administering to a subject a composition comprising paclitaxel (taxol), phyllanthoside or vincristine.

The invention also provides a method of treating renal cell carcinoma comprising administering to a subject a composition comprising mithramycin or a pharmaceutically acceptable salt or solvate thereof and further comprising rapamycin or a pharmaceutically acceptable salt or solvate thereof.

The “subject” can include domesticated animals, such as cats, dogs etc., livestock (e.g. cattle, horses, pigs, sheep, goats, etc.), laboratory animals (e.g. mouse, rabbit, rat, guinea pig etc.) and birds. Preferably, the subject is a mammal such as a primate, and more preferably a human.

The composition may further comprise a pharmaceutically acceptable carrier. Suitably the composition is administered in amount that is effective to treat renal cell carcinoma in a subject. In general an “effective amount” of a compound is that amount needed to achieve the desired result or results.

A composition comprising a compound of the instant invention may be administered to a subject by any of a number of routes of administration including, for example, orally (for example drenches as in aqueous or non-aqueous solutions or suspension, tablets, boluses, powders, granules, pastes for application to the tongue); sublingually, anally, rectally, or vaginally (for example as a pessary, cream or foam); parenterally (including intramuscularly, intravenously, subcutaneously or intrathecally as for example a sterile solution or suspension); nasally; intraperitoneally; subcutaneously; transdermally (for example as a patch applied to the skin); or topically (for example as a cream, ointment or spray applied to the skin). The compound may also be formulated for inhalation.

In particular, the invention provides a method of treating clear cell renal cell carcinoma comprising administering to a subject a composition comprising mithramycin. The invention also provides a method of treating clear cell renal cell carcinoma comprising administering to a subject a composition comprising mithramycin and further comprising rapamycin.

The invention also provides a method of treating renal cell carcinoma associated with Von Hippel-Lindau disease or Birt-Hogg-Dubé syndrome comprising administering to a subject a composition comprising mithramycin. The invention also provides a method of treating renal cell carcinoma associated with Von Hippel-Lindau disease or Birt-Hogg-Dubé syndrome comprising administering to a subject a composition comprising mithramycin and further comprising rapamycin.

The invention also provides a method of inhibiting the growth of FLCN-null renal cell carcinoma cells comprising administering to a subject a composition comprising mithramycin. The invention also provides a method of inhibiting the growth of FLCN-null renal cell carcinoma cells comprising administering to a subject a composition comprising mithramycin and rapamycin.

Also provided is a method of inhibiting the growth of VHL-null renal cell carcinoma cells comprising administering to a subject a composition comprising mithramycin. Also provided is a method of inhibiting the growth of VHL-null renal cell carcinoma cells comprising administering to a subject a composition comprising mithramycin and rapamycin.

The invention also provides a method of differentially inhibiting the growth of FLCN-null cells over FLCN-wild type cells comprising administering a composition comprising mithramycin. The invention also provides a method of differentially inhibiting the growth of FLCN-null cells over FLCN-wild type cells comprising administering a composition comprising mithramycin and rapamycin.

Although ChA3 shows discriminate killing of VHL-deficient cells in ccRCC cell lines, we have discovered that ChA3 does not show differential growth inhibitory activity in FLCN-deficient and FLCN-wild type cell lines. We did not find any differences between the growth inhibitory activity of chromomycin A3 (ChA3) to FLCN deficient and positive cells.

However, it has surprisingly been found that mithramycin demonstrates selected sensitivity of FLCN-null over FLCN-wild type cells. Mithramycin demonstrates around a 10-fold difference in the GI₅₀ values between FLCN-null cells and FLCN-wild type cells (i.e. the GI₅₀ for mithramycin for FLCN-wild type cells was almost 10 times more than that for FLCN-null cells), and preferentially induces caspase 3/7 activity in FLCN-null cells in a dose dependent manner. It is almost 10-fold more cytotoxic to FLCN-null cells than wild type FLCN expressing cells.

Surprisingly, it has also been found that mithramycin exhibits differential growth inhibitory activity according to VHL status in isogenic VHL-null and VHL-expressing ROC cell lines.

The activity of mithramycin is in contrast to ChA3, for which we did not find any differences between the growth inhibitory activity of FLCN deficient and FLCN positive cell lines. We have found that mithramycin demonstrates selective cytotoxic activity for FLCN-null over FLCN-wild type cells. Therefore, mithramycin presents a treatment for RCC, in particular as a genotypic selective therapy for FLCN-deficient RCC and VHL-deficient RCC.

Also, it has surprisingly been found that Paclitaxel (taxol) demonstrated an almost 7-fold difference in the GI₅₀ values between UOK257 FLCN-null and UOK257 FLCN-wild type cells. Phyllanthoside and vincristine both demonstrated around a 2-fold difference in the GI₅₀ values between UOK257 FLCN-null and UOK257 FLCN-wild type cells (see Table 1). This shows that paclitaxel, phyllanthoside and vincristine present a treatment for renal cell carcinoma associated with FLCN inactivation.

The invention will now be described by way of example with reference to the figures, in which:

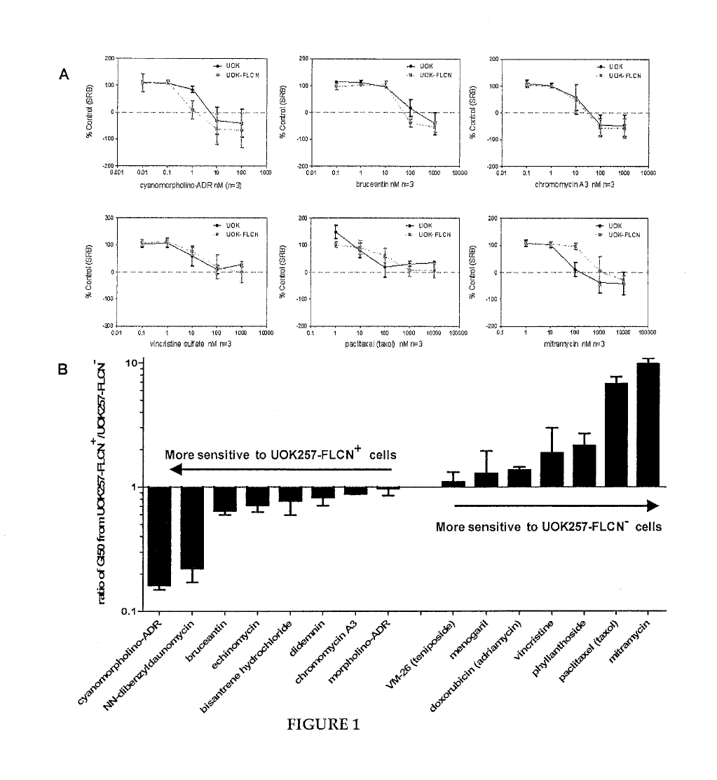

FIG. 1A shows the inhibition of UOK257-FLCN-negative and UOK257-FLCN-positive cell growth by 6 compounds, as determined by the SRB assay.

FIG. 1B shows the ratio of GI₅₀ for UOK257-FLCN-negative and UOK257-FLCN-positive cells for 15 compounds.

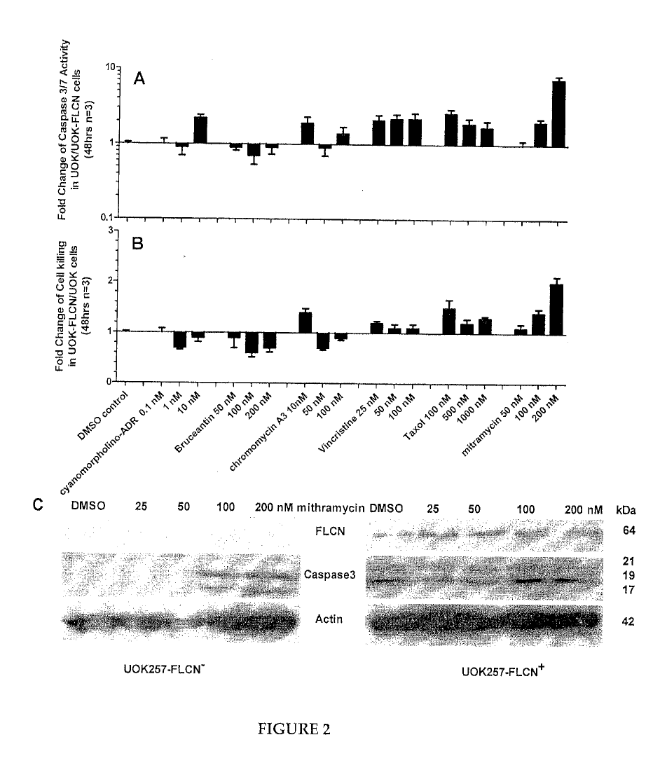

FIG. 2A shows the ratio of Caspase3/7 activity for UOK257-FLCN-negative and UOK257-FLCN-positive cells for 6 compounds.

FIG. 2B shows the ratio of cell viability for UOK257-FLCN-negative and UOK257-FLCN-positive cells in response to drug exposure.

FIG. 2C shows cellular protein expression in UOK cells with and without FLCN expression in response to mithramycin exposure.

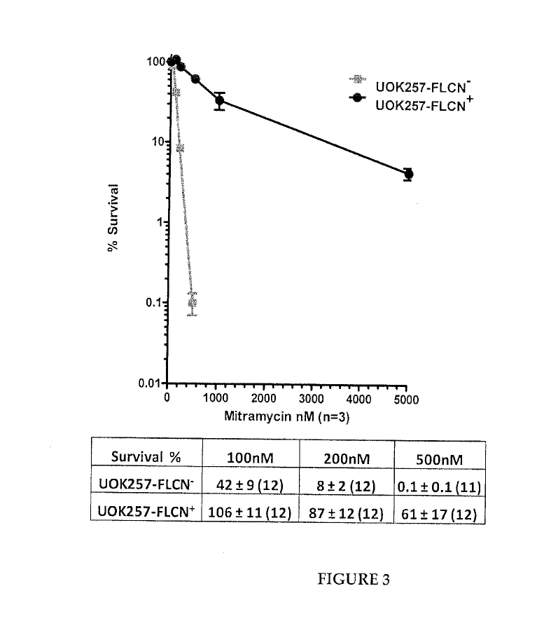

FIG. 3 shows the cytotoxicity of mithramycin as measured by clonogenic assay in UOK257-FLCN-negative and UOK257-FLCN-positive cells.

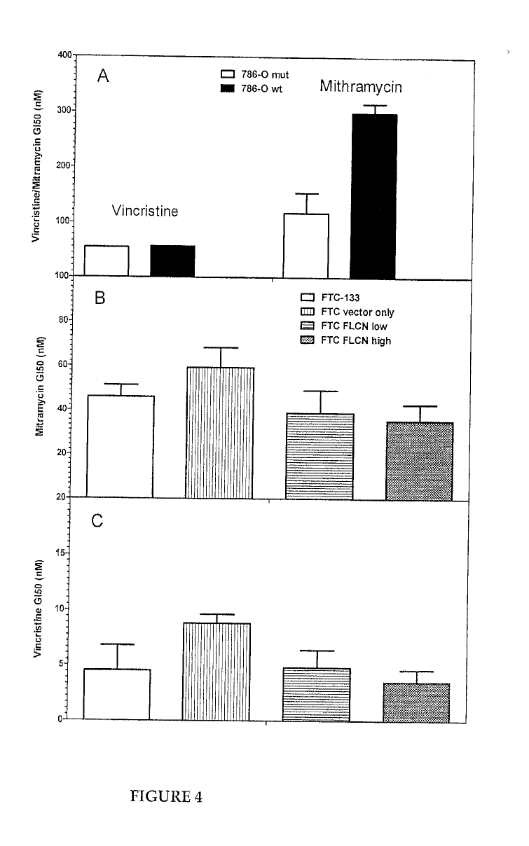

FIG. 4 shows the GI₅₀ values for the inhibition of 786-O and FTC-133 cell growth by mithramycin.

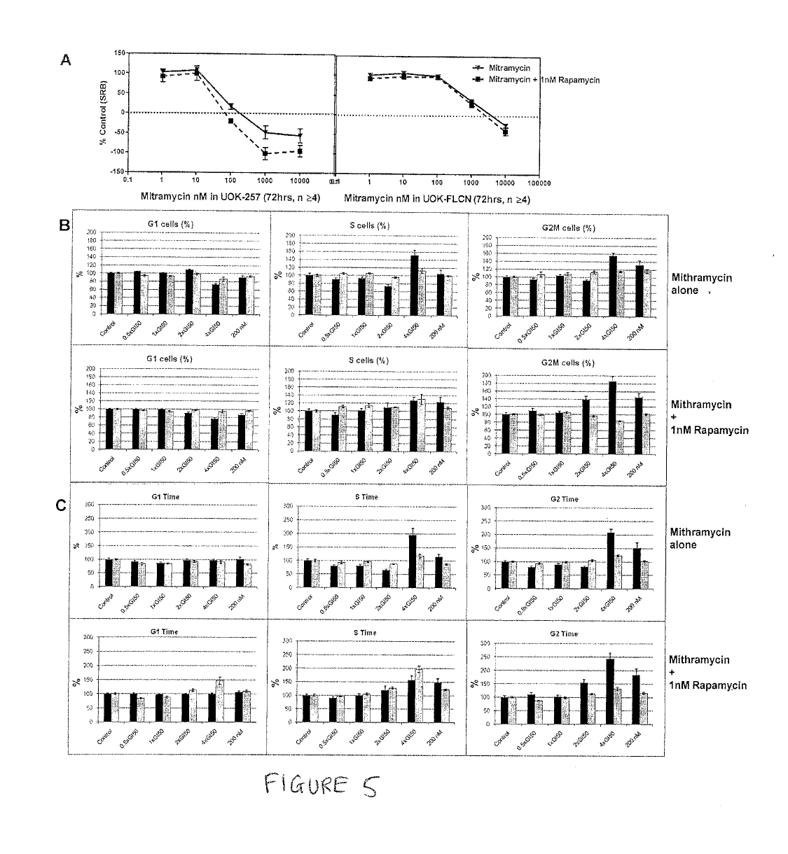

FIG. 5 shows cell growth inhibition of UOK257-FLCN⁻ and UOK257-FLCN⁺ determined by the SRB assay in response to exposure of mithramycin alone and mithramycin in combination with rapamycin.

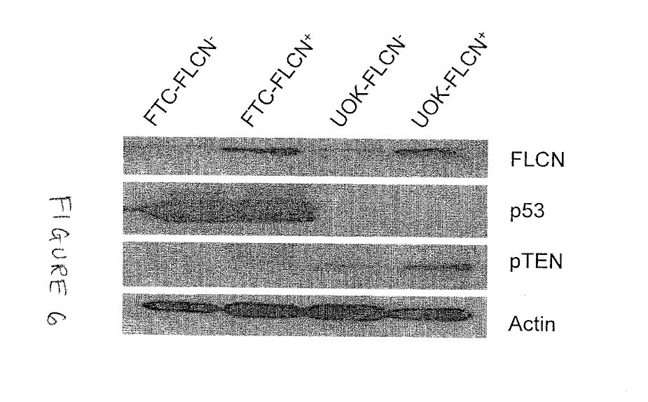

FIG. 6 shows basal level of cellular protein expression in UOK and FTC cells with and without FLCN expression.

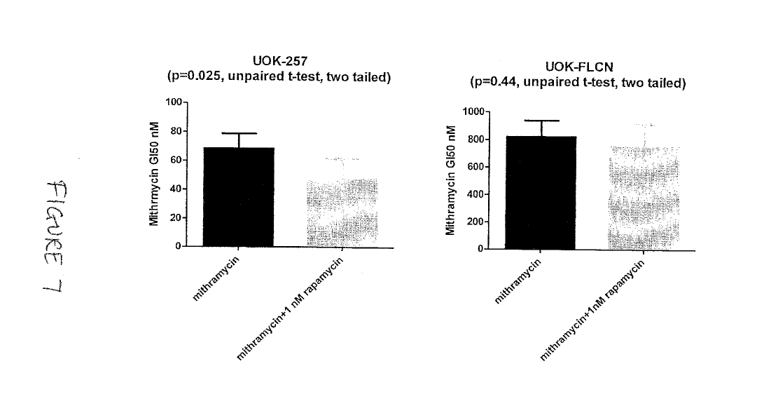

FIG. 7 shows an unpaired t-test comparing UOK-257 and UOK-FLCN cells incubated in mithramycin only and mithramycin in combination with rapamycin.

### EXAMPLES

We performed experiments to demonstrate the genotype selective cytotoxicity of mithramycin in RCC cell lines with FLCN inactivation and VHL inactivation and compared this with other possible anti-cancer agents.

### Materials:

Morpholino-ADR (NSC 354646), cyanomorpholino-ADR (NSC 357704), echinomycin (NSC 13502), chromomycin A3 (NSC 58514), bruceantin (NSC 67574), vincristine sulfate (NSC 165563), didemnin B (NSC 325319), paclitaxel (Taxol, NSC 125973), mithramycin (NSC 24559), phyllanthoside (NSC 266492), bisantrene hydrochloride (NSC 337766), doxorubicin (Adriamycin, NSC 123127), VM-26 (teniposide, NSC 122819), menogaril (NSC 269148), N,N-dibenzyldaunomycin (NSC 268242), and rapamycin (NSC 226080) were provided by the Developmental Therapeutics Program of the National Cancer Institute (NCI)/NIH (http://dtp.nci.nih.gov).

### Cell Lines and Cell Culture:

Human renal carcinoma of BHD origin cells UOK-257 (UOK-FLCN⁻) and FLCN transfected UOK-257 cells UOK-FLCN⁺ (Yang et al Cancer Genet Cytogenet. 2008 Jan. 15; 180(2):100-9) were provided by Dr Marston Linehan and Dr. Laura S Schmidt (Urologic Oncology Branch, Centre for Cancer Research, National Cancer Institute, National Institutes of Health, Bethesda, Md. 20892, USA). UOK-257 is the only RCC cell line that has been derived from a patient with BHD and harbours a germline FLCN frameshift mutation (c.1285dupC) (predicted, in the absence of nonsense mediated mRNA decay, to lead to premature protein truncation (p.His429ProfsX27) (Yang et al., Cancer Genet Cytogenet, 2008 Jan. 15; 180(2):100-9). We are not aware of any sporadic RCC cell lines with homozygous FLCN inactivation. FTC-133 cells were purchased from ECACC (Salisbury, United Kingdom). These cells are a non-RCC cell line derived from human thyroid carcinoma. 786-0 cells were available from the author's lab (Clifford et al., Hum Mol Genet. 2001 May 1; 10(10):1029-38). 786-O is a kidney cancer cell line that harbours an inactivating VHL gene mutation (c.311delG p.G104fs*55). All cell lines were cultured in DMEM with supplement of 10% foetal bovine serum except for FTC-133 cells which were incubated in medium with DMEM and Hens (1:1).

### Growth Inhibition Assay:

The sensitivity of the cell lines to drug-induced cell growth inhibition was determined using the sulphorhodamine B assay (SRB) as described by Lu et al., Clin Cancer Res. 2001:7:2114-23. Briefly, adherent exponentially growing cells were seeded into 96-well plates at 3-5×10³ cells/100 μl/well. After 20-24 h at 37° C., drugs were added at the appropriate drug concentrations to the wells (final DMSO conc. 1%) as indicated in the Results section. After drug treatment for 72 hours, the cells were fixed in situ by adding equal volume of Carnoy's fixative (methanol:acetic acid=3:1), washed, air dried and stained with sulphorhodamine B (0.4%, Sigma-Aldrich, Poole, United Kingdom). The absorbance per well was measured at 570 nm on a Victor X3 Multilabel Plate Reader (PerkinElmor, Beaconsfield, United Kingdom).

### Caspase-Glo 3/7 Assay and Cell Viability Assay:

The ability to induce caspase 3/7 activation after exposure to compounds was measured by Caspase-Glo 3/7 Assay (Promega, Southampton, United Kingdom) according to manufacturer's instruction. The cells were seeded and dosed as described in growth inhibition assay. At the end of incubation, 70 μl of medium was removed and 30 μl of assay reagent was added to the remaining medium. Additional one hour incubation with shaking was carried out at room temperature. The resultant luminescent light was measured in a Victor X3 Multilabel Plate Reader (PerkinElmer, Beaconsfield, United Kingdom). Cell viability after compound exposure was determined by CellTiter-Blue Cell Viability Assay (Promega, Southampton, the United Kingdom) according to manufacturer's instruction. At the end of drug treatment, 20 μl of reagent was added to the medium in 96 wells and the cells were incubated for additional 4 hours. The cell viability was measured by fluorescence with a 570 nm excitation and 590 nm emission set in a Victor X3 Multilabel Plate Reader (PerkinElmer, Beaconsfield, United Kingdom).

### Clonogenic Cell Survival Assay:

The cytotoxicity of mithramycin was determined in UOK-257 cells with/without FLCN expression. Exponentially growing cells were seeded into 100-mm Petri dishes at densities ranging from 250 to 1×10⁵ cells/dish, the cell seeding density being adjusted to give an estimated 10-300 colonies/dish following drug exposure. The cells were left to attach for 24 h and freshly made mithramycin was added at the appropriate concentrations to the dishes. The final concentration of DMSO in the medium was 1%. Four dishes with two seeding densities for each drug concentration were used and at least three experiments were carried out under each set of conditions. The cells were exposed to mithramycin for 72 hours, after which the medium was aspirated, the dishes were washed once with warm PBS, and fresh drug-free medium was added. The cells were incubated for an additional 10-16 days until visible colonies appeared, which were fixed with Carnoy's fixative (methanol:acetic acid 3:1, v:v), and visualized by staining cells in sulphorhodamine B (0.4%). Colonies with over 30 cells were counted. Cloning efficiencies of untreated cells were: UOK257-FLCN⁻ 26% and UOK257-FLCN⁺34%. Cell survival following drug exposure was expressed as % control cloning efficiency or survival.

### Western Blot Analysis:

Cellular protein expression in UOK Cells with/without FLCN expression in response to drug exposure was determined using total cell extracts at 48 h after treatment, according to standard procedures. Protein concentration was determined using DC protein assay kit according to the manufacturer's instructions (Bio-Rad Laboratories Ltd, Hertfordshire, United Kingdom). Twenty p1 of protein from each sample was electrophoresed on 12.5% (w/v) SDS-PAGE gels and electroblotted on to nitrocellulose membrane (Amersham Pharmacia Biotech UK Ltd., Buckinghamshire, United Kingdom). Antibody against FLCN (a gift from Prof. Arnim Pause, Rosalind and Morris Goodman Cancer Centre Montreal, Canada), caspase3 (Cell Signalling Technology, Hertfordshire, United Kingdom), and actin (Sigma-Aldrich Company Ltd., Dorset, United Kingdom) were used. The signal was detected using the enhanced chemiluminescence (ECL+Plus, Amersham) system after addition of anti-mouse IgG-HRP conjugate (DAKO, Ely, United Kingdom).

### Cell Cycle Analysis

UOK-257 cells with/without FLCN expression were seeded at 2,000 cells per well in 96-well plates either with 0.1% DMSO or with 1 nmol/L Rapamycin and incubated at 37° C./5% CO2 overnight to adhere. The drugs were added at the appropriate concentrations to the wells in replicates of 8 as indicated in the Results section. At the end of the time course, media was carefully removed and cells were fixed in 85% ice-cold ethanol. After removal of ethanol, cells were incubated in the dark at 37° C. for 20 minutes in PBS buffers containing 0.1% Triton X-100, 100 mg/mL RNase A and 10 mg/mL Propidium iodide (PI). The 96-well plates were subsequently scanned using the Acumen eX3 cytometer (TTP LabTech).

### Results:

### Identification of Candidate Drugs by COMPARE Algorithm Based on FLCN Expression Patterns on the NCI-60 Panel:

A tissue-directed anticancer drug screen was created in 1990 by the NCI to evaluate compounds for antineoplastic activity. A cell line panel consisting of 60 tumour cell lines from nine different tissue types deemed the NCI60 was established. Importantly, these 60 cell lines span a spectrum of molecular defects, allowing for the analysis of drug activity with respect to specific molecular alterations. These cell lines have been characterised extensively for a range of attributes including microarray gene expression profiles and in vitro drug sensitivity (e.g. the cell lines have been screened for cytotoxic sensitivity for >14,000 compounds) (see NCI Developmental Therapeutic Program, http://dtp.nci.nih.gov). To identify candidate anticancer agents that might differentially affect cancer cell lines with differing levels of FLCN expression, cell lines were classified according to published microarray data as high, medium or low FLCN expressors and analysed with the COMPARE algorithm (Ross et al., Nat Genet. 2000; 24:227-35). These FLCN expression categories were used to construct a theoretical drug activity pattern, or seed pattern, reflective of a drug that targets FLCN deficient cells, or more specifically FLCN low expressors. The aim was to identify drugs to which cell lines with low FLCN expression were most sensitive and cell lines with high FLCN expression were most resistant (cells with medium or undetermined FLCN expression were designated as a neutral sensitivity). The COMPARE algorithm was used to compare the FLCN seed pattern with individual compounds in the Developmental Therapeutics Program (DTP) database (http://dtp.nci.nih.gov) and 15 compounds were selected with the similar sensitivity pattern to the FLCN seed (the compounds selected are listed in Table 1 below).

Screen Compound Sensitivities in UOK-257 Cells with/without FLCN Expression:

UOK-257 is the only RCC cell line that has been derived from a patient with BHD and harbours a germline FLCN frameshift mutation (c.1285dupC) (predicted, in the absence of nonsense mediated mRNA decay, to lead to premature protein truncation (p.His429ProfsX27) (Yang et al Cancer Genet Cytogenet, 2008 Jan. 15; 180(2):100-9). Both UOK257-FLCN⁻ and UOK257-FLCN⁺ cells were examined for their sensitivities to growth inhibition induced by 15 compounds selected from the COMPARE analysis. Various concentrations of compounds were introduced into the cells 24 hrs after seeding, incubation was continued for 72 hours and growth inhibition was assessed using the suiphorhodamine B (SRB) assay. GI₅₀ values, defined as the concentration of compounds required to inhibit growth by 50%, were then calculated. The ratio of GI₅₀ value from UOK257-FLCN⁺ and UOK257-FLCN⁻ were calculated and used as an indicator for the sensitivity to FLCN mutant cells.

FIG. 1(A) shows inhibition of UOK257-FLCN⁻ (solid line), and UOK257-FLCN⁺ (dotted line) cell growth by cyanomorpholino-ADR, bruceantin, mopholino-ADR, vincristine, taxol and mithramycin as determined by the SRB assay. Cells were exposed continuously to the indicated concentrations of drugs for 72 hours. Points and GI₅₀ values are the mean±SD from at least three experiments. FIG. 1(B) shows the ratio of GI₅₀ from UOK257-FLCN⁺ and UOK257-FLCN⁻ in all 15 compounds. The ratios were calculated and used as indicators for the sensitivity to mutant FLCN cells.

As shown in FIG. 1 and Table 1, seven compounds were relatively more inhibitory to UOK257-FLCN⁻ cells (ratio>1) and the most selective growth-inhibitory sensitivity was induced by mithramycin with almost 10-fold difference in the GI₅₀ values between UOK257-FLCN⁻ cells (64.2±7.9 nM; n=3) and UOK257-FLCN⁺ cells (634.3±147.9 nM; n=4).

Whereas the UOK257-FLCN⁻ cells were less sensitive to the other eight compounds (ratio<1) and cyanomorpholino-ADR induced the least growth inhibitory sensitivity with 6-fold difference in the GI₅₀ values between UOK257-FLCN⁺ cells (0.62±0.16 nM; n=3) and UOK257-FLCN⁻ cells (3.79±0.39 nM; n=3).

The data indicated that although these compounds were selected from COMPARE algorithm with the same criteria, there was a broad variation between the differential sensitivity of the cells with/without FLCN expression to different drugs.

TABLE 1
Compounds-induced growth inhibition in
UOK cells with/without FLCN^{a}

| | | | GI₅₀ Ratio of |
|---|---|---|---|
| | | | UOK- |
| | | | FLCN⁺/ |
| | GI₅₀ nM | GI₅₀ nM | UOK- |
| Compounds | in UOK-FLCN⁻ | in UOK-FLCN⁺ | FLCN⁻ |
| morpholino-ADR | 0.45 ± 0.03 (3) | 0.44 ± 0.10 (3) | 0.97 |
| cyanomorpholino- | 3.79 ± 0.39 (3) | 0.62 ± 0.16 (3) | 0.16 |
| ADR | | | |
| echinomycin | 5.72 ± 0.22 (3) | 4.03 ± 0.89 (3) | 0.71 |
| didemnin B | 7.94 ± 1.66 (4) | 6.53 ± 1.78 (4) | 0.82 |
| vincristine | 30.1 ± 10.3 (5) | 56.4 ± 17.5 (5) | 1.88 |
| sulphate | | | |
| chromomycin A3 | 37.4 ± 6.9 (3) | 33.0 ± 5.7 (3) | 0.88 |
| mithramycin | 64.2 ± 7.9 (3) | 634 ± 148 (4) | 9.88 |
| bruceantin | 68.4 ± 14.9 (3) | 43.6 ± 5.1 (3) | 0.64 |
| paclitaxel (taxol) | 75.5 ± 9.2 (5) | 514 ± 86 (3) | 6.81 |
| phyllanthoside | 199 ± 79 (5) | 427 ± 97 (4) | 2.15 |
| doxorubicin | 423 ± 54 (3) | 581 ± 109 (4) | 1.37 |
| (adriamycin) | | | |
| menogaril | 588 ± 57 (4) | 757 ± 117 (4) | 1.29 |
| VM-26 (teniposide) | 773 ± 162 (3) | 849 ± 358 (3) | 1.10 |
| bisantrene | 1753 ± 288 (6) | 1345 ± 602 (5) | 0.77 |
| hydrochloride | | | |
| N,N- | 5739 ± 918 (4) | 1273 ± 549 (4) | 0.22 |
| dibenzyldaunomycin | | | |

^{a}Data are mean ± SE with the number of the experiments given in parenthesis.
GI₅₀ values were calculated by fitting a sigmoidal concentration/inhibition curve to the results using non-linear least square regression (GraphPad PRISM ™) to data generated by the SRB assay.

Induction of Caspase3/7 Activity and Cell Death by NCI Compounds in UOK-257 Cells with/without FLCN Expression:

Six compounds (UOK257-FLCN⁺-inhibitory: cyanomorpholino-ADR, bruceantin, chromomycin A3 and UOK257-FLCN⁻-Inhibitory: mithramycin, taxol and vincristine) were selected for further investigation for their ability to induce Caspase3/7 activity in the pair of UOK cell lines. After 48 hrs incubation, with three concentration ranges for each drug, the capase3/7 activity was detected by amount of luminescence for each cell line. The ratio of caspase3/7 activity from UOK257-FLCN⁻ and UOK257-FLCN⁺ were calculated and used as indicators for the ability of the individual compound to induce caspase3/7 activity to FLCN mutant cells.

FIG. 2(A) shows the ratio of caspase3/7 activity from UOK257-FLCN⁻ and UOK257-FLCN⁺ in response to exposure of cyanomorpholino-ADR, bruceantin, mopholino-ADR, vincristine, taxol and mithramycin. These ratios were calculated and used as indicators for the sensitivity to mutant FLCN cells.

As shown in FIG. 2A, three UOK257-FLCN⁺-inhibitory compounds displayed a low degree of differential caspase3/7 activity induction in UOK257-FLCN⁺ cells over parent UOK257-FLCN⁻ cells, especially in bruceantin (ratio<1) at all three concentrations.

However, there was no concentration dependent increase of caspase3/7 activity observed. Whereas all three UOK257-FLCN⁻ sensitive compounds showed at least 2-fold induction of caspase3/7 activity, in which only mithramycin induced a concentration dependent increase with at least 8-fold higher caspase3/7 activity in UOK257-FLCN⁻ cells than in UOK257-FLCN⁺ cells (200 nM, 48 hrs). Cell viability assay was another method to examine whether the degree of cell death corresponded to cell growth inhibition and of active caspase3/7 induction.

FIG. 2(B) shows the ratio of cell viability from UOK257-FLCN⁺ and UOK257-FLCN⁻ in response to the same drugs exposure. These were calculated and used as indicators for the sensitivity to mutant FLCN cells. Cells were exposed continuously to the indicated concentrations of drugs for 48 hours. Values are the mean±SD from at least three experiments.

As shown in FIG. 2B, both UOK257-FLCN⁺ and UOK257-FLCN⁻-inhibitory compounds showed a very similar pattern to those in growth inhibition assay (FIG. 1), although at a lower degree (up to 2-fold change). However, only mithramycin induced cell death in a concentration-dependent manner (2-fold, 200 nM, 48 hrs), a very similar pattern for induction of caspase3/7 activity by mithramycin.

Western blot analysis was performed to examine the change of cellular caspase3 protein expression after treatment of mithramycin for 48 hours in both UOK257-FLCN⁻ and UOK257-FLCN⁺ cells.

FIG. 2(C) shows cellular protein expression in UOK cells with/without FLCN expression in response to mithramycin exposure. Exponential growing cells were exposed continuously to mithramycin for 48 hours and then western blotting was carried out by using antibodies as described in “Materials and Methods”.

As shown in FIG. 2C, the appearance of active forms (17 and 19 kDa) of cleaved caspase3 were found at 100 and 200 nM, but not in low concentrations of mithramycin (25 and 50 nM) in treated UOK257-FLCN⁻ cells (which was corresponded to the induction of caspase3/7 activity shown in FIG. 2A).

However, in UOK257-FLCN⁺ cells two higher molecular weight active forms (19 and 21 kDa) of caspase3 were present throughout in non-treated and mithramycin-treated cellular lysates at the concentrations examined. It was interesting to note that a high molecular weight caspase 3 band (21 kDa) was found both in UOK257-FLCN⁻ and UOK257-FLCN⁺ cells whereas the 19 kDa active form of caspase 3 was present in the cellular lysates of UOK257-FLCN⁺ irrespective of mithramycin treatment. The time course of mithramycin treatment in UOK257-FLCN⁻ cells showed that the cleaved active caspase3 bands appeared as early as 24 hrs, peaked at 48 hrs and lasted for 72 hrs (data not shown).

Comparison of Mithramycin-Induced Cytotoxicity in UOK Cells with/without FLCN Expression:

Potential mechanisms underlying the differences in sensitivity to mithramycin in regard to FLCN expression within the cells were further investigated. Clonogenic assays were carried out to examine whether the mithramycin-induced differential effects in proliferation, caspase3/7 activation and cell death reflected to mithramycin-induced cytotoxicity measured by clonogenic cell survival. After exposure to mithramycin at various concentrations for 72 hrs UOK257-FLCN⁻ and its counterpart UOK257-FLCN⁺ cells were then incubated in drug-free medium until colonies were formed.

FIG. 3 shows the cytotoxicity of mithramycin as measured by clonogenic assay in UOK257-FLCN⁻ and UOK257-FLCN⁺ cells. Cells were exposed to mithramycin at the indicated concentrations for 72 hours and then placed in drug-free medium for an additional 10-15 days. Error bars represent the range of values obtained from 3 experiments.

As shown in FIG. 3, at a concentration of 200 nM less than 8% viable cells were observed for UOK-257 cells without FLCN expression.

In contrast, more than 85% cell survival was counted in UOK257-FLCN⁺ cells, which was a 10-fold difference between two cell lines. At a higher concentration of 500 nM less than 0.1% UOK257-FLCN⁻ cells survived whereas more than 60% UOK257-FLCN⁺ cells were able to form colonies. Even at the highest examined concentration of 5000 nM, still more than 4% UOK257-FLCN⁺ cells survived. The data are consistent with the hypothesis that mithramycin was preferentially cytotoxic for UOK-257 cells without FLCN expression.

The differential effects between two cell lines in response to mithramycin exposure were further studied statistically. There were strong correlations between mithramycin-induced growth inhibition and cytotoxicity (r²=0.98, p=0.009), and cell death (r²=0.91, p=0.047) for these cell lines. In addition there was 8-fold increase in caspase3/7 activity in UOK257-FLCN⁻ over UOK257-FLCN⁺ cells at 200 nM of mithramycin (FIG. 2A). All those data suggested that FLCN mutation and loss of function in UOK257 cells may confer a greater mithramycin sensitivity measured by these methods.

Comparison of Mithramycin Alone or in Combination of Rapamycin in UOK257 Cells with/without FLCN Expression

Aberrant mTOR signaling has been reported to be associated with FLCN/Flcn inactivation in human cell lines and in transgenic mice. Hence, we proceeded to investigate whether inhibition of the mTOR pathway by rapamycin influenced the differential mithramycin sensitivity found in UOK cells with/without FLCN expression. Preliminary data (not shown) showed that a low concentration of rapamycin (1 nmol/L) inhibited mTOR activity (as measured by S6R phosphorylation) but had only a small effect (10%-15% reduction) on cell proliferation. Both UOK257-FLCN⁻ and UOK-FLCN⁺ cells were incubated in the presence or absence of 1 nmol/L rapamycin overnight before adding mithramycin at various concentrations for 72 hours and GI₅₀ values were determined from growth inhibition curves. Rapamycin at 1 nmol/L can potentiate mithramycin sensitivity by 1.5-fold in UOK257-FLCN⁻ cells (69±10 nmol/L, n=6 for mithramycin and 47±14 nmol/L, n=4 for mithramycin and rapamycin) suggesting that mithramycin in combination of rapamycin was more potent than mithramycin alone in the cells (P=0.025, unpaired t test—FIG. 7). This is shown in FIG. 5A. Although there was a slight difference (1.1-fold) in UOK257-FLCN⁺ cells, the combination drugs-induced growth inhibition was not as profound (P=0.44, unpaired t test—FIG. 7) as seen in UOK257-FLCN⁻ cells when higher mithramycin concentrations were applied (see FIG. 5).

FIG. 5 shows cell growth inhibition of UOK257-FLCN⁻ cells and UOK257-FLCN⁺ cells determined by the SRB assay in response to exposure of mithramycin alone (solid line) and mithramycin in combination of 1 nmol/L rapamycin (dotted line) for 72 hours (A). After UOK257-FLCN cells (black bar) and UOK257-FLCN⁺ cells (pale bar) were incubated with mithramycin alone or in combination of 1 nmol/L rapamycin for 48 hours, the percentage of each cell cycle population (B) and of each cell cycle time (C) were calculated. Cells were incubated with either DMSO or 1 nmol/L rapamycin overnight an then exposed continuously to carious concentrations of mithramycin. At the end of incubation, cells were fixed and stained with PI as indicated in Material and Methods before being scanned with the Acumen eX3 cytometer.

To study the underlining mechanism of differential sensitivities of mithramycin or in combination with rapamycin in 2 cell lines, cell cycle analysis was carried out using cytometry (Acumen Explorer). To compare cell cycle change at the same degree of mithramycin inhibition, mithramycin was applied in a range of GI₅₀ concentrations (ranging from 0.5-fold to 4-fold) to both UOK257-FLCN and UOK-FLCN⁺ cell lines in the presence/absence of 1 nmol/L rapamycin. In the absence of 1 nmol/L rapamycin, there was 60% Increase in cell population of both S and G2-M phases in comparison with DMSO control in UOK257-FLCN cells (4×GI₅₀ mithramycin, 48 hours)—see FIG. 5B. However, only 15% increase in S and G2-M phases was found in UOK-FLCN⁺ cells at 4×GI₅₀ mithramycin [a 10-fold higher concentration (2.5 mmol/L) than that used in its counterpart UOK257-FLCN⁻ cells (0.25 mmol/L)]. In the presence of 1 nmol/L rapamycin, a more pronounced increase in G2-M phase was observed at a lower mithramycin concentration (40% at 2×GI₅₀ and 90% at 4×GI₅₀) in UOK257-FLCN⁻ cells, indicating that rapamycin potentiates the cell cycle inhibitory effect (G2-M arrest) of mithramycin. Moreover, the cell cycle inhibition was associated with lengthening of the S and G2-M time in UOK257-FLCN⁻ cells (100% at 4×GI₅₀ mithramycin alone; FIG. 5C). The effect of mithramycin on the G2-M time was also potentiated by rapamycin (70% at 2×GI₅₀, 250% at 4×GI₅₀; FIG. 5C). In contrast, the modest effect of mithramycin on the G2-M phase in UOK-FLCN⁺ cells was not substantially influenced by the presence of rapamycin (25% at 4×GI₅₀). In summary, in response to mithramycin exposure, UOK257-FLCN⁻ cells were arrested in the S and G2-M phases of the cell cycle and low dose of rapamycin (1 nmol/L) strongly potentiated mithramycin sensitivity by promoting the G2-M arrest.

Comparison of Mithramycin Sensitivity in Non-RCC Cells and Gene Status of p53, PTEN in the Cells Examined

Mithramycin sensitivity was also examined in a non-RCC cell lines (FTC-133 a human thyroid carcinoma) with/without FLCN expression. A FLCN containing construct was introduced into parental FTC-133 cells and stably transfected cells were selected. SRB growth inhibition assay was carried out in cells transfected with empty vector and low FLCN and high FLCN expression. It was interesting to note that there was no apparent differential mithramycin sensitivity to FTC-133 cells with different levels of FLCN expression (59.3±8.9 nmol/L, n=5 for empty vector; 38.7±10.4 nmol/L, n=3 for FLCN expressed at low levels; and 35.3±7.3 nmol/L, n=5 for FLCN expressed at high levels). Similarly, there was no selective vincristine sensitivity in FTC-133 cells with different FLCN levels (data not shown).

We next investigated the p53 and PTEN status of the UOK-257 and FTC-133 cell lines. Direct genomic sequencing and Western blot were used to investigate gene mutation status and protein expression levels. In addition to inactivation of FLCN gene, both cell lines also harbored p53 gene mutations (Table 2).

TABLE 2
Gene status in human tumor cell lines

| Cell line | UOK-257 | UOK 257 | FTC-133 | FTC-133 |
|---|---|---|---|---|
| Cancer type | BHD - codon | BHD - amino acid | Thyroid - | Thyroid - amino |
| | change | change | codon | acid change |
| | | | change | |
| FLCN | c.1285dupC | p.His429ProfxX27 | c.1285delC | p.His429ThrfsX39 |
| p53 | c.153C > T | p.Gln52X | c.818G > A | p.Arg273His |
| PTEN | Wt/wt | | c.388C > T | Arg130X |

A c.153C>T mutation resulted in an early truncation of p53 protein in UOK-257 and a point mutation of p53 gene leaded to inactivation of p53 function by accumulation of the protein in FTC-133 cells (FIG. 6). It is interesting to note that there was PTEN gene mutation resulting in a truncated protein in FTC-133 cells whereas wild type PTEN was present in the UOK-257 cell line.

FIG. 6 shows basal level of cellular protein expression in UOK and FTC cells with/without FLCN Expression. Western blotting was carried out by using antibodies as described in Materials and Methods.

### Comparison of Mithramycin-Sensitivity in VHL-Mutant RCC 786-0 Cells and Other Non-RCC Cells:

We then investigated whether the differential cytotoxicity of mithramycin according to FLCN expression observed in UOK-257 RCC cell lines might also extend to cell lines with differing expression of the VHL TSG. Growth inhibition assays of mithramycin were undertaken in a pair of isogenic renal carcinoma cells 786-0 with/without VHL expression.

FIG. 4 shows GI₅₀ values from inhibition of 786-0 (A) and FTC-133 (B and C) cell growth by mithramycin and vincristine as determined by the SRB assay. Cells were exposed continuously to various concentrations of drugs for 72 hours. GI₅₀ values were determined as the concentrations at 50% growth inhibition from at least three experiments.

An almost 3-fold greater growth Inhibitory sensitivity for mithramycin was observed in 786-O cells with mutant VHL (117±37 nM, n=3) compared to the same cell line expressing wt VHL (299±17 nM, n=3, FIG. 4A). However, there was no significant change in vincristine treatment for 786-0 cells with/without VHL expression. The data suggested that mithramycin may sensitize RCC cells deficient in FLCN and VHL.

Mithramycin sensitivity was also examined in a non-RCC cell line (FTC-133 a human thyroid carcinoma) with/without FLCN expression. A FLCN containing construct was introduced into parental FTC-133 cells and stably transfected cells were selected. SRB growth inhibition assay was carried out in cells transfected with empty vector and low FLCN and high FLCN expression. It was interesting to note that there was no apparent differential mithramycin sensitivity to FTC-133 cells with different levels of transfected FLCN expression (59.3±8.9 nM, n=5 for empty vector; 38.7±10.4 nM, n=3 for FLCN expressed at low levels; and 35.3±7.3 nM, n=5 for FLCN expressed at high levels; FIG. 4B). Similarly, there was no selective vincristine sensitivity in FTC-133 cells with different FLCN levels (FIG. 4B).

### CONCLUSIONS

We detected selective sensitivity of UOK257 FLCN-null over UOK257 FLCN-wild type cells for seven compounds. Most notably, mithramycin demonstrated a ˜10-fold difference in the GI₅₀ values between UOK257 FLCN-null and UOK257 FLCN-wild type cells, preferentially induced caspase 3/7 activity in UOK257 FLCN-null cells in a dose dependent manner and was almost 10-fold more cytotoxic to UOK257 FLCN-null cells than UOK257 FLCN-wild type expressing cells.

We found that mithramycin exhibited differential growth inhibitory activity (though less marked than in UOK257 FLCN deficient and positive cell lines) according to VHL status in isogenic VHL-null and VHL-expressing 786-O RCC cell lines.

To date, UOK-257 is the only RCC cell line available that is derived from a patient with BHD and we are not aware of any sporadic RCC cell lines with homozygous FLCN inactivation. In our hands, knockdown of FLCN expression by siRNA in sporadic RCC with wild-type FLCN was incomplete and so was an unsuitable model for replicating the findings in UOK257-FLCN null and expressing cell lines. In addition, such an approach would not replicate folliculin inactivation as the initiating event in tumorigenesis. We note that previously, using a similar strategy to one that we employed to identify candidate agents that might show differential activity between high and low FLCN expressing cancer cell lines, Sutphin et al. used the COMPARE algorithm to identify 10 compounds that showed differential activity against VHL low and high expressing NCI60 cell lines. Four of these compounds were tested against paired VHL null and VHL expressing RCC cell lines

Chromomycin A3 (ChA3) was found to exhibit differential toxicity, in clonogenic survival studies, to VHL-deficient cell lines relative to VHL-positive RCC cell lines (Sutphin et al., 2007 Cancer Res. 2007 Jun. 15; 67(12):5896-90525). ChA3 is an aureolic acid compound that binds DNA in the minor groove and inhibits transcription. However, although it would appear that mithramycin can exhibit genotypic differential cytotoxicity according to both FLCN and VHL expression status, we did not find any differences between the growth inhibitory activity of ChA3 to FLCN deficient and positive UOK-257 cell lines.

The activity of mithramycin is in contrast to ChA3, for which we did not find any differences between the growth inhibitory activity in FLCN deficient and positive UOK257 cell lines.

Mithramycin binds to GC-rich regions and inhibits the transcription of genes with GC-rich promoters and has been used to treat several types of cancer, including testicular carcinoma and leukaemia (Yuan et al., Cancer 2007; 110:2682-2690. Inhibition of Sp1 activity has been implicated in mithramycin cytotoxicity, but we did not find any consistent differences between the mithramycin-induced changes in expression of Sp1 target genes between FLCN deficient and positive UOK257 cell lines. Furthermore, siRNA knockdown of Sp1 in FLCN-deficient and positive UOK257 cell lines did not differentially inhibit cell growth or induce caspase 3/7 activity.

In response to mithramycin exposure, UOK257-FLCN⁻ cells were mainly arrested and blocked in S and G2-M cell cycle and low dose of rapamycin (1 nmol/L) potentiated mithramycin sensitivity (1.5-fold in G2-M population and 2-fold in G2-M period time, 2×GI₅₀, 48 hours). It was reported that p53 status played an important role in modulation of mithramycin-induced cell polyploidy and cell death in colon carcinoma cells. Mithramycin SK, a novel analog of mithramycin, results in polyploidization and mitotic catastrophe in HCT116 cells with wt p53 and most cell populations died by necrosis, whereas HCT-116 (p53^{-/-}) cells died mainly from G2-M block through early p53-independent apoptosis. These observations led us to speculate that in response to mithramycin exposure, UOK-257 cells with inactivated p53 gene might also die through a p53-independent apoptosis pathway when arrested in G2-M block. In FLCN-replete UOK257 cells; however, little G2-M cell cycle block was induced by mithramycin.

RCC from BHD patients may show increased HIF-2 expression and Sutphin et al. found that overexpression of HIF-2 in VHL-positive clear cell RCC cell lines phenocopied the effect of VHL inactication on susceptibility to ChA3 toxicity.

The observation that mithramycin demonstrates genotypic selective cytotoxicity to both FLCN and VHL-deficient cell lines might suggest that these effects result from shared characteristics of FLCN and VHL-deficient cell lines. However, the observation that ChA3 does not demonstrate selective toxicity to FLCN-deficient and positive UOK257 cell lines suggests that other factors are, at least in part, implicated.

Mithramycin does not show genotypic selective growth inhibitory activity in a non-renal cancer cell line (FTC-133). There are several possible explanations for this observation. Firstly, whereas FLCN inactivation is an initiating event in BHD RCC (e.g. in UOK257 cells) and VHL inactivation is thought to be the earliest tumourigenic event in sporadic clear cell RCC, FLCN inactivation in the FTC-133 thyroid carcinoma cell line may have occurred at a late stage of tumourigenesis and so might not be of critical functional importance. Secondly, the functional consequences of tumour suppressor gene inactivation may differ according to cell tissue type (e.g. VHL inactivation or hypoxic induction of HIF-2 expression induces oncogenic CCND1 expression in RCC cell lines but CCND1 expression is not hypoxia-inducible in non-RCC cancer cell lines (Bindra et al., Cancer Res. 2002 Jun. 1; 62(11):3014-9; Zatyka et al., Cancer Res. 2002 Jul. 1; 62(13):3803-11), possibly explaining the very restricted cancer susceptibility phenotypes seen in inherited cancer syndromes such as VHL disease and BHD syndrome (we note that renal cancer, but not thyroid cancer, is a major complication of BHD suggesting that folliculin has a gatekeeper role in the renal but not thyroid cells). Thirdly the response of a FLCN-deficient cell line to mithramycin treatment may be modified by co-existing mutations (or epimutations) in additional tumour suppressor genes/oncogenes (e.g. FTC-133 also harbours mutations in TP53 and PTEN).

The findings that mithramycin demonstrates differential inhibition of growth of FLCN-null cells over FLCN-wild type cells demonstrate that mithramycin provides a genotypic selective therapy for FLCN deficient RCC. Furthermore, the findings that mithramycin demonstrates differential inhibition of growth of VHL-null cells over VHL-wild type cells demonstrates that mithramycin provides a treatment for renal cell carcinoma (and in particular clear cell renal cell carcinomas).

These findings herein demonstrate that mithramycin can be used to treat renal cell carcinoma, and in particular renal cell carcinoma associated with Von Hippel-Lindau disease or Birt-Hogg-Dubé syndrome.

### SUMMARY

Brit-Hogg-Dubé (BHD) syndrome, an autosomal dominant familial cancer, is associated with increased risk of kidney cancer. BHD syndrome is caused by loss-of-function mutations in the folliculin (FLCN) protein. To develop therapeutic approaches for renal cell carcinoma (RCC) in BHD syndrome, we adopted a strategy to identify tumor-selective growth inhibition in a RCC cell line with FLCN inactivation. The COMPARE algorithm was used to identify candidate anticancer drugs tested against the NCI-60 cell lines that showed preferential toxicity to low FLCN expressing cell lines. Fifteen compounds were selected and detailed growth inhibition (SRB) assays were done in paired BHD RCC cell lines (UOK257 derived from a patient with BHD). Selective sensitivity of FLCN-null over FLCN-wt UOK257 cells was observed in seven compounds. The most selective growth-inhibitory sensitivity was induced by mithramycin, which showed an approximately 10-fold difference in GI₅₀ values between FLCN-null (64.2±7.9 nmol/L, n=3) and FLCN-wt UOK257 cells (634.3±147.9 nmol/L, n=4). Differential ability to induce caspase 3/7 activity by mithramycin was also detected in a dose-dependent manner. Clonogenic survival studies showed mithramycin to be approximately 10-fold more cytotoxic to FLCN-null than FLCN-wt UOK257 cells (200 nmol/L). Following mithramycin exposure, UOK257-FLCN-null cells were mainly arrested and blocked in S and G2-M phases of the cell cycle and low dose of rapamycin (1 nmol/L) potentiated mithramycin sensitivity (1.5-fold in G2-M population and 2-fold in G2-M period time, 2×GI₅₀, 48 hours). These results provide a basis for further evaluation of mithramycin as a potential therapeutic drug for RCC associated with BHD.

1. A composition comprising mithramycin or a pharmaceutically acceptable salt or solvate thereof for use in the treatment of renal cell carcinoma.

2. A composition according to claim 1 for use in the treatment of clear cell renal cell carcinoma.

3. A composition according to claim 1 for use in the treatment of renal cell carcinoma associated with Von Hippel-Lindau disease or Birt-Hogg-Dubé syndrome.

4. A composition according to claim 3 for use in the treatment of renal cell carcinoma associated with Birt-Hogg-Dubé syndrome.

5. A composition according to claim 1 for use in the inhibition of growth of FLCN-null renal cell carcinoma cells.

6. A composition according to claim 1 for use in the inhibition of growth of VHL-null renal cell carcinoma cells.

7. A composition according to claim 1 for use in the differential growth inhibition of FLCN-null cells over FLCN-wild type cells.

8. A composition according to claim 1 for use in the treatment of renal cell carcinoma associated with FLCN inactivation.

9. A composition according to claim 1 for use in the treatment of renal cell carcinoma associated with VHL inactivation.

10-12. (canceled)

13. A composition according to claim 1 wherein the composition further comprises rapamycin or a pharmaceutically acceptable salt or solvate thereof.

14. (canceled)

15. A method of treating renal cell carcinoma comprising administering to a subject a composition comprising mithramycin or a pharmaceutically acceptable salt or solvate thereof.

16. A method according to claim 15, wherein the renal cell carcinoma is associated with Birt-Hogg-Dubé syndrome or Von Hippel-Lindau disease.

17. A method according to claim 15, wherein the method comprises the step of inhibiting the growth of FLCN-null renal cell carcinoma cells.

18. A method according to claim 15, wherein the method comprises the step of differentially inhibiting the growth of FLCN-null cells over FLCN-wild type cells.

19. A method according to claim 15, wherein the composition further comprises rapamycin or a pharmaceutically acceptable salt or solvate thereof.

20. A method of inhibiting the growth of FLCN-null renal cell carcinoma cells in a subject, the method comprising administering to the subject a composition comprising mithramycin or a pharmaceutically acceptable salt thereof.

21. A method of inhibiting the growth of VHL-null renal cell carcinoma cells in a subject, the method comprising administering to the subject a composition comprising mithramycin or a pharmaceutically acceptable salt thereof.
